SEQUENCE LISTING (8 sequences)

SEQ ID NO: 1 - DNA, 2113 bp, Eikenella corrodens
atgaagaacatcttattaggctgtggtcataaggaacttggtgactatctgaagagcttgattgaaactttggaaaagggtggtcacaccatccgaatagcccatgacccgcaagaaatcctgacgttcctgaaacatgacgccagaataggctcagtgctttgtacactggatattttcaaccgagaattagacgaacaaattattgcactcaacgatgagctacccgtattcattctgaagcccaccgactgcgacaaacccgtggacttcggcgctgtaggcgaccatgccaccttcatcgattgccatctgttcagcaacgaagacgttgttgataaaattgaaaaagcaatctgtcactatatcgacaatatcaccccgcccttcactaaagccctgtttgactacgttgataaaaacaaatacacattctgtactccgggtcacatgagcggtaccgctttcttgaaatctcccgtaggtagcctgttctacgacttctacggtgaaaacacattcaaatcagatatttcagtttctatgggcgaattgggctctctgctcgaccactccggcccgcacaaagaggccgaagaatacatcgccgaaaccttcaatgccgaccacagctacatcgtaaccaacggtacttccaccgccaacaaaatcgtgggtatgtactccgttccggccggcagcaccgtactgatcgaccgtaactgccacaaatcattaacccacctgttgatgatgagcgacattactccggtgtatctgaaaccgacccgtaacgcctacggtatcttgggtggtattccgcaaaaagaattcaccaaagaagttattaccgaaaaacttaccaaagttccgggcgccacttggccggtacatgccgtgattaccaactccacttatgacggcctgttctacaacaccgacaagatcaaagacaccctggatgtgaaatccatccacttcgactctgcttgggttccctacaccaacttcagcccgatctacaacggcaaaaccggtatgggcggtaagcaggttaaagacaaggtaatctttgaaacccactctacccacaaactgctggccgcattctcacaggcttccatgattcacgttaagggtaacctgaacaccgccaccttcggcgaagcctacatgatgcacacctccacttctccgttctaccccatggtagcctctactgaagtggcagcagccatgatgcgcggcaactccggtaaacgcctgatgcaggattcactggaacgcgccgttaaattccgtaaggaaatcaagaaacacaaagcccatgccgactcatggtatttcgatgtatggcaacctgaaaatgttgacaacatcgaatgctgggaattgcatcagaccgacaaatggcacggcttcaaagacatcgacgcccagcacatgtacctcgacccgattaaggtaactctgcttaccccgggcttggataaaaacggcgagctggaaaaaaccggtatccccgccaacctcgtttccaaattcttggaagaccgcggcatcatcgtggagaaaaccggcccctacaacatcctcgtgctcttcagcatcggcgtggacgacaccaaagccctcagcctgctgcacgcattgaacgagttcaaatccctgtacgatgccaacgccaccgtagaagaagtattgccgcgcgtattcaacgaatcgccctctttctaccaagaaatgcggattcaagaattggcacaaggcatccacagcctgatctgcaaacacaacctgcctgaactgatgttcagcgcattcgaagtgttgccgaccatggtgatgaacccgcacaaagccttccagcttgagctgaaaggtcagattgaagactgctacttggaagacatggtaggcaagatcaacgccaacatgattctgccctatcctcccggcgtaccgttggtaatgcctggtgaaatgatcaccgaagaaagcaagcccatcttggaattcctgatgatgctgtgcgaaatcggcgcccacttccccggcttcgaaaccgacatccacggtgcttaccgtcaagaagacggccgctataaagtta

SEQ ID NO: 2 - protein, 709 aa, Eikenella corrodens
MKNILLGCGHKELGDYLKSLIETLEKGGHTIRIAHDPQEILTFLKHDARIGSVLCTLDIFNRELDEQIIALNDELPVFILKPTDCDKPVDFGAVGDHATFIDCHLFSNEDVVDKIEKAICHYIDNITPPFTKALFDYVDKNKYTFCTPGHMSGTAFLKSPVGSLFYDFYGENTFKSDISVSMGELGSLLDHSGPHKEAEEYIAETFNADHSYIVTNGTSTANKIVGMYSVPAGSTVLIDRNCHKSLTHLLMMSDITPVYLKPTRNAYGILGGIPQKEFTKEVITEKLTKVPGATWPVHAVITNSTYDGLFYNTDKIKDTLDVKSIHFDSAWVPYTNFSPIYNGKTGMGGKQVKDKVIFETHSTHKLLAAFSQASMIHVKGNLNTATFGEAYMMHTSTSPFYPMVASTEVAAAMMRGNSGKRLMQDSLERAVKFRKEIKKHKAHADSWYFDVWQPENVDNIECWELHQTDKWHGFKDIDAQHMYLDPIKVTLLTPGLDKNGELEKTGIPANLVSKFLEDRGIIVEKTGPYNILVLFSIGVDDTKALSLLHALNEFKSLYDANATVEEVLPRVFNESPSFYQEMRIQELAQGIHSLICKHNLPELMFSAFEVLPTMVMNPHKAFQLELKGQIEDCYLEDMVGKINANMILPYPPGVPLVMPGEMITEESKPILEFLMMLCEIGAHFPGFETDIHGAYRQEDGRYKVKIVKA

SEQ ID NO: 3 - DNA, 1345 bp, Staphylococcus epidermidis
atgaaaagaccaataattcaaaaattaaatcacttgatagagaaaaaagctatctctatgcatgttcctggacataaaaacatgacaatcggctacttaaataggcttgatttagcaatggatatgacagaaattactggattagatgatatgcattatcctgaaggaattattttagaaagcatggagaattttaggaaacataaaaactatgatgctttcttattagttaacggaacgacttcaggtatattatcggttatccaagcgttttcgacaagaaaaggtaaatatttaattagtagaaatgttcataaatcagtatttcacggtttagacataacacaacaacaagcgacaataactaagacagatgtcagtaagaaaacgaatcaatatgtaaatccaaagataaatcaagataaaaatcaatattataaacttgccatctgtacataccctaattattacggtgaaacttttgatatttctcaatatatcaaacaattacatcacagagggataccgatattagtagatgaagcgcatggtgcacattttggtttagatggatttccagaatcctcaatgaattttaatgctgattacgttgtgcagtcatatcacaaaacactccctgcactaacaatgggatcagttatatatatacataaagatgcaccattaagacaacaagtcatagattatttaacttatttccaaacgtcaagtccttcgtacctcattatgtctagtttagaattagcgaataaattttataaagaatatgattctacattatttgaccaacgaagaaagatgttaattgatttattagtaaatatgggatttacagttatagaaccagaggatcctttaaaattggttgtgagttttgagggtgttgaaggttatgatgtgcaaaaatggtttgaggataaagaaatttatgtagaattagctgatatgtatcaagtgttactcgttctccccctatggcatgaaggagataaatttccttttaagttgttgattgaaaaaattagagaaattaacgtgccaaaaaaatgtacgcgcgacataaaacctcttaattttatgacgggttttagcgaatacaaaactgttcattttcaaaatacgaaagaagtgtctattaaaagggcagaaggtaaagttttagcacaacatatcgttccataccctccaggtataccggtgatgtttaaaggagaagtcgtgacgtctcatatgatagacttattaaataaatatgataaacaaaatattaaagttgaaggtttaaatcataaaaaaatattagttaaggatgaataagaaatgt

SEQ ID NO: 4 - protein, 445 aa, Staphylococcus epidermidis
MKRPIIQKLNHLIEKKAISMHVPGHKNMTIGYLNRLDLAMDMTEITGLDDMHYPEGIILESMENFRKHKNYDAFLLVNGTTSGILSVIQAFSTRKGKYLISRNVHKSVFHGLDITQQQATITKTDVSKKTNQYVNPKINQDKNQYYKLAICTYPNYYGETFDISQYIKQLHHRGIPILVDEAHGAHFGLDGFPESSMNFNADYVVQSYHKTLPALTMGSVIYIHKDAPLRQQVIDYLTYFQTSSPSYLIMSSLELANKFYKEYDSTLFDQRRKMLIDLLVNMGFTVIEPEDPLKLVVSFEGVEGYDVQKWFEDKEIYVELADMYQVLLVLPLWHEGDKFPFKLLIEKIREINVPKKCTRDIKPLNFMTGFSEYKTVHFQNTKEVSIKRAEGKVLAQHIVPYPPGIPVMFKGEVVTSHMIDLLNKYDKQNIKVEGLNHKKILVKDE

SEQ ID NO: 5 - DNA, 22 bp, Eikenella corrodens
gggaattcatatgaagaacatc

SEQ ID NO: 6 - DNA, 26 bp, Eikenella corrodens
cagttggatccgcgtgggttaagctt

SEQ ID NO: 7 - DNA, 18 bp, Staphylococcus epidermidis
gggaattcatatgaaaag

SEQ ID NO: 8 - DNA, 22 bp, Staphylococcus epidermidis
cagttggatcttattcatcctt

(19) United States
(12) Patent Application Publication
(10) Pub. No.: US 2008/0213305 A1
(43) Pub. Date: Sep. 4, 2008

(54) Vaccine for periodontitis and methods of use

(76) Inventors: Martin Levine, Norman, OK (US); James Haddock, Newcastle, OK (US)

Correspondence Address: DUNLAP CODDING & ROGERS, P.C., PO BOX 16370, OKLAHOMA CITY, OK, 73113, US

(21) Appl. No.: 11/999,821

(22) Filed: Dec. 7, 2007

Related U.S. Application Data
(60) Provisional application No. 60/873,890, filed on Dec. 8, 2006.

(51) Int. Cl. A61K 39/116

(52) U.S. Cl. 424/203.1

(57) ABSTRACT

The present invention is directed to an immunogenic composition for inducing an immune response against lysine decarboxylase for treating or preventing periodontitis in a subject. The present invention is also directed to methods for inducing an immunogenic response in a subject by administering such immunogenic composition.

### CROSS-REFERENCE TO RELATED APPLICATIONS

The present application claims the benefit under 35 U.S.C. 119(e) of U.S. Provisional Application Ser. No. 60/873,890, filed Dec. 8, 2006. The entirety of which is hereby expressly incorporated herein by reference.

### STATEMENT REGARDING FEDERALLY SPONSORED RESEARCH OR DEVELOPMENT

Some aspects of this invention were made in the course of Grant 1 R21 DE 14583-01 awarded by the National Institute of Dental Research Exploratory/Development Research and therefore the Government has certain rights in some aspects of this invention.

### BACKGROUND OF THE INVENTION

Periodontal disease is an irreversible loss of tooth attachment that is usually associated with gingival inflammation (gingivitis) and plaque adherent to teeth. Gingivitis is detected by sulcular swelling, redness and bleeding on gentle probing, or the exudation of fluid onto absorbent paper. The fluid is the gingival crevicular fluid (GCF), the amount of which is a measure of mild inflammation (sub-clinical gingivitis) which associates with plaque accumulation on clean teeth after oral hygiene is abolished. The loss of tooth attachment (periodontitis) is apparent as cementum exposed beneath the cementoenamel junction within deepened sulci (pockets) or in the oral cavity directly (recession). Severe periodontitis culminates in loose teeth, a major problem in older humans and animals. The animals eat poorly, the infection induces deleterious systemic effects throughout the body, and they become difficult to handle and expensive to treat.

A large and complex group of organisms that develop by utilizing GCF components as substrates are associated with periodontitis progression. Although Porphyromonas gingivalis is considered a key organism, it only dominates a small number of untreated periodontal pockets and may be completely absent. Various gram negative bacteria cause periodontitis in the absence of P. gingivalis. In dogs with periodontitis, P. gingivalis is absent, but various other black pigmented anaerobic bacteria are present. Although the oral location of plaque lends itself to mucosal immunization, antibodies in saliva have limited access to periodontal pockets which are bathed in GCF containing IgG and IgM antibodies. Pfizer has reported a subcutaneously administered vaccine to a mixture of Porphyromonas spp., a ‘Porphyromonas Denticanis-Gulae-Salivosa Bacterin.’ In the mouse oral cavity, the ‘Bacterin’ inhibited colonization by the component organisms and the development of experimental periodontitis compared with sham-immunized mice. A 1-year conditional license was issued by the USDA Center for Veterinary Biologics in July 2005 (Notice 05-15), but as yet there are no reports of this vaccine being commercially available despite Pfizer's announcement that it would be available in June 2006. If Pfizer's ‘Bacterin’ is being tested in the soft diet beagle dog model and if it inhibits the growth of the target bacteria, other gram negative bacteria would grow on the GCF instead and little or no effect on gingivitis and periodontitis would be detected in a less artificial model than the mouse model. This very well could be why Pfizer does not have their vaccine on the market and why they have not been pushing their vaccine at veterinary conferences during 2006.

Periodontitis results in a higher systemic level of C-reactive protein, IL-6, and neutrophils. These elevated inflammatory factors may increase inflammation in atherosclerotic lesions, potentially increasing the risk for cardiac or cerebrovascular events. Other findings suggest that intensive periodontal treatment reduces these and other systemic inflammatory markers. It also reduces systolic BP, and improves lipid profiles with subsequent changes in cardiovascular risk when compared to standard therapy.

Advanced human periodontal disease is a common problem in many developing countries, where it often results in multiple tooth loss by early middle age. Giving the vaccine in early adolescence before periodontitis has developed may enhance the quality of life for people for whom periodontal treatment cannot easily be obtained. Therefore, availability of a vaccine for preventing or modulating periodontal disease in humans, dogs, and other mammals would be of great benefit in both developed and developing countries.

### BRIEF DESCRIPTION OF THE DRAWINGS

FIG. 1: Detection of lysine decarboxylase protein by immune goat serum. Biorad Minigel and blotter used in 12% sodium dodecyl-polyacrylamide gel electrophoresis (SDS-PAGE) followed by PVDF blotting, as suggested by the manufacturer. E. corrodens (1.7 μg), or Biofilm extract (16.2 μg) were added to wells indicated. Blots were either stained for protein (Prot.) or blocked and reacted with pre-immune or immune goat serum used for the experiment in FIG. 2. Arrow indicates 80 kDa protein identified as lysine decarboxylase.

FIG. 2: Effect of immune or pre-immune goat serum on cadaverine production by E. corrodens or plaque extracts. Symbols: Unimmunized goat (or pre-immune serum) □-□; Immunized goat □-□ and □-□. Inhibition was retained for up to 9 months if immune serum was not frozen, but simply stored at 4° C. Goats were immunized with 0.25 mg E. corrodens extract in Freunds adjuvant.

FIG. 3: Detection of recombinant lysine decarboxylases from E. corrodens (left side) and S. epidermidis (right side) expressed in E. coli lysates. Top: Protein stained; bottom immunoblotted. Lysates are prepared as described in section 34. 12% polyacrylamide gels are run and blots prepared as described in the legend to FIG. 1, except that rabbit antibodies described in section 00028 below were used to detect S. epidermidis antigen instead of goat antibodies. Goat and rabbit antibodies were used diluted 1 to 25,000. Reaction of the IgG with antigen was detected using anti-goat or anti-rabbit IgG alkaline phosphatase conjugated second antibody as recommended by the manufacturer (Sigma Chemical Co., St Louis Mo.).

FIG. 4: Immunization of dogs with E. corrodens lysine decarboxylase extract. Four dogs were immunized subcutaneously in the back of the neck every two weeks with 1 ml of 15% Rehydragel adjuvant (section 00043 below) containing 0.2 mg of E. corrodens extract in 65 mM NaCl. Two sham-immunized controls received the Rehydragel containing 65 mM NaCl without the lysine decarboxylase protein. To detect the antibodies, wells in Immulon 4 HBX-Extra High Binding 96-Well Plates (Thermo-Fisher Scientific, 81 Wyman Street Waltham, Mass. 02454) were coated with 0.1 ml of 50 mM sodium carbonate buffer containing 10 ug/ml of the protein in E. corrodens extract. The coated wells were blocked with albumin, washed and then reacted with serial 4-fold dilutions of dog serum beginning at 1 to 50 and using 1% bovine serum albumin, 0.05% Tween 20 in PBS pH 7.0 as diluent. Wells were washed, reacted with dog anti-IgG alkaline phosphatase conjugate, diluted 1:1,000 in PBS containing 0.5% bovine serum albumin, washed again, and developed with ρ-nitrophenyl phosphate tablets. These procedures were as recommended by the manufacturer of the second antibody conjugate (Sigma Chemical Co., St Louis Mo.). Immunized dogs: Filled triangles and squares. Sham-immunized dogs (no protein in the Rehydragel): Unfilled circles and squares.

### DETAILED DESCRIPTION OF THE INVENTION

Before explaining at least one embodiment of the invention in detail by way of exemplary drawings, experimentation, results, and laboratory procedures, it is to be understood that the invention is not limited in its application to the details of construction and the arrangement of the components set forth in the following description or illustrated in the drawings, experimentation and/or results. The invention is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2nd ed., Cold Spring Harbor Laboratory Press, Cold Spring Harbor, N.Y. (1989) and Coligan et al. Current Protocols in Immunology (Current Protocols, Wiley Interscience (1994)), which are incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

As used herein, the term “nucleic acid segment” and “DNA segment” are used interchangeably and refer to a DNA molecule which has been isolated free of total genomic DNA of a particular species. Therefore, a “purified” DNA or nucleic acid segment as used herein, refers to a DNA segment which contains a coding sequence isolated away from, or purified free from, unrelated genomic DNA, genes and other coding segments. Included within the term “DNA segment”, are DNA segments and smaller fragments of such segments, and also recombinant vectors, including, for example, plasmids, cosmids, phage, viruses, and the like. In this respect, the term “gene” is used for simplicity to refer to a functional protein-, polypeptide- or peptide-encoding unit. As will be understood by those in the art, this functional term includes genomic sequences, cDNA sequences or combinations thereof. “Isolated substantially away from other coding sequences” means that the gene of interest forms the significant part of the coding region of the DNA segment, and that the DNA segment does not contain other non-relevant large portions of naturally-occurring coding DNA, such as large chromosomal fragments or other functional genes or DNA coding regions. Of course, this refers to the DNA segment as originally isolated, and does not exclude genes or coding regions later added to, or intentionally left in, the segment by the hand of man.

Preferably, DNA sequences in accordance with the present invention will further include genetic control regions which allow the expression of the sequence in a selected recombinant host. The genetic control region may be native to the cell from which the gene was isolated, or may be native to the recombinant host cell, or may be an exogenous segment that is compatible with and recognized by the transcriptional machinery of the selected recombinant host cell. Of course, the nature of the control region employed will generally vary depending on the particular use (e.g., cloning host) envisioned.

Truncated genes also fall within the definition of preferred DNA sequences as set forth above. Those of ordinary skill in the art would appreciate that simple amino acid removal can be accomplished, and the truncated versions of the sequence simply have to be checked for the desired biological activity in order to determine if such a truncated sequence is still capable of functioning as required. In certain instances, it may be desired to truncate a gene encoding a protein to remove an undesired biological activity, as described herein.

Nucleic acid segments having a desired biological activity may be isolated by the methods described herein. The term “a sequence essentially as set forth in SEQ ID NO:X” means that the sequence substantially corresponds to a portion of SEQ ID NO:X and has relatively few amino acids or codons encoding amino acids which are not identical to, or a biologically functional equivalent of, the amino acids or codons encoding amino acids of SEQ ID NO:X. The term “biologically functional equivalent” is well understood in the art and is further defined in detail herein, as a gene having a sequence essentially as set forth in SEQ ID NO:X, and that is associated with the ability to perform a desired biological activity in vitro or in vivo.

The DNA segments of the present invention encompass DNA segments encoding biologically functional equivalent proteins and peptides. Such sequences may arise as a consequence of codon redundancy and functional equivalency which are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques, e.g., to introduce improvements to the enzyme activity or to antigenicity of the protein or to test mutants in order to examine biological activity at the molecular level or to produce mutants having changed or novel enzymatic activity and/or substrate specificity.

The term “polypeptide” as used herein is a generic term to refer to native protein, fragments, or analogs of a polypeptide sequence. Hence, native protein, fragments, and analogs are species of the polypeptide genus.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “recombinant” in the context of polypeptide coding regions and the polypeptides encoded by such coding regions refers to non-native products wherein the coding regions, and typically the expression thereof, have been manipulated in vitro by man to differ from their occurrence in nature. The polypeptides utilized in the methods of the present invention may be produced in a number of different recombinant systems known in the art, including but not limited to, archeal, prokaryotic, or eukaryotic systems. For expression in an appropriate expression system, the desired viral capsid polypeptide coding regions are operably linked into an expression vector and introduced into a host cell to enable expression. The coding region with the appropriate regulatory regions will be provided in proper orientation and reading frame to allow for expression. Methods for gene construction are known in the art. See, in particular, Molecular Cloning, A Laboratory Manual, Sambrook et al, eds., Cold Spring Harbor Laboratory, Second Edition, Cold Spring Harbor, N.Y. (1989) and the references cited therein.

As used herein, when the term “purified” is used in reference to a molecule, it means that the concentration of the molecule being purified has been increased relative to molecules associated with it in its natural environment. Naturally associated molecules include proteins, nucleic acids, lipids and sugars but generally do not include water, buffers, and reagents added to maintain the integrity or facilitate the purification of the molecule being purified. For example, even if mRNA is diluted with an aqueous solvent during oligo dT column chromatography, mRNA molecules are purified by this chromatography if naturally associated nucleic acids and other biological molecules do not bind to the column and are separated from the subject mRNA molecules.

As used herein, when the term “isolated” is used in reference to a molecule, the term means that the molecule has been removed from its native environment. For example, a polynucleotide or a polypeptide naturally present in a living animal is not “isolated,” but the same polynucleotide or polypeptide separated from the coexisting materials of its natural state is “isolated.” Further, recombinant DNA molecules contained in a vector are considered isolated for the purposes of the present invention. Isolated RNA molecules include in vivo or in vitro RNA replication products of DNA and RNA molecules. Isolated nucleic acid molecules further include synthetically produced molecules. Additionally, vector molecules contained in recombinant host cells are also isolated. Thus, not all “isolated” molecules need be “purified.”

“Treatment” refers to both therapeutic treatment and prophylactic or preventative measures. Those in need of treatment include those already with the disorder as well as those in which the disorder is to be prevented.

When the terms “one,” “a,” or “an” are used in this disclosure, they mean “at least one” or “one or more,” unless otherwise indicated.

Numerous aspects and advantages of the invention will be apparent to those skilled in the art upon consideration of the following detailed description which provides illumination of the practice of the invention.

Periodontitis is associated with the formation of plaque on the teeth. If young adults with minimal gingivitis, no periodontitis, and freshly cleaned teeth abstain from oral hygiene, plaque adheres to teeth at the gingival sulcus and thickens within 12-24 hours. The plaque is composed of viridans streptococci, Actinomyces spp., and small amounts of E. corrodens and Capnocytophaga spp. Over the next 2-4 days, gram negative fusobacteria, other anaerobic rods and spirochetes attach and start to grow. If oral hygiene remains inadequate, the proportion of gram negative bacteria and spirochetes increases further over the next few months. These anaerobic bacteria metabolize amino acids to ammonia and short chain fatty acids. Ammonia creates an alkaline environment, and the short chain fatty acids are cytotoxic and associated with persistent gingivitis. The alkaline environment causes calcium phosphate to precipitate as calculus, sheltering these gram negative bacteria and their products. Gingivitis is maintained and it predisposes to the host changes that cause periodontitis.

The structure of the tooth-epithelial attachment is a unique factor that leads to plaque thickening when oral hygiene is abolished. The attachment is comprised of undifferentiated keratinocytes forming a continuous basal cell layer adherent to the teeth and gingival stroma. These layers and their underlying laminar membranes are continuous at their apical extremity above the gingival collagen fibers. The entire epithelium is enclosed by these two basal layers and permeable to interstitial fluid, which provides nutrients required by the proliferative basal keratinocytes cells comprising the tooth adherent basal layer which has no underlying capillaries. E. corrodens appears in human plaque within a few hours of abolishing oral hygiene and it secretes lysine decarboxylase that converts lysine to cadaverine and carbon dioxide. Lysine becomes depleted from the interstitial fluid, and tooth-attached cell proliferation at the junctional epithelial coronal extremity is inhibited. The affected cells release mediators that attract GCF to the sulcus by the same route as the interstitial fluid transudate. Traces of GCF are present in sulci that have minimal gingivitis, and if oral hygiene is abolished, the amount increases markedly. The GCF contains serum proteins that are absent from interstitial fluid, and, because of its faster flow, it provides more glucose, free amino acids and vitamins.

The nutrients in GCF enable the gram positive bacteria to thicken and provide a more anaerobic environment for gram negative bacterial attachment. The GCF replenishes lysine, causing the attached cells to stop signaling until lysine again becomes depleted. This repeated cycle of GCF induction eventually permits enough gram negative microbiota to overgrow the gram positive bacteria at the base of a sulcus where the GCF first appears in an oral cavity. Eventually gingivitis becomes clinically apparent and its persistence changes the host response to cause periodontitis. In humans, repeated tooth brushing stops the overgrowth of gram negative bacteria, but not the lysine depletion cycles. In addition, tooth brushing will not remove any calculus-protected bacteria. In dogs, brushing is rarely practicable, but their hard diet controls plaque until calculus develops.

Lysine decarboxylase converts lysine to cadaverine and carbon dioxide in mammalian cell culture fluid at physiological pH and starves mammalian cells of lysine in vitro. Because cadaverine is only derived from lysine, the cadaverine fraction of the lysine plus cadaverine content of a plaque sample is a measure of its lysine decarboxylase enzyme activity. The mean mass of plaque assayed and its content of lysine plus cadaverine (nmol) per mg wet plaque, were found to be similar in subjects irrespective of the absence, presence or severity of periodontitis. Nevertheless, the cadaverine fraction was significantly greater in subjects who had been treated for periodontitis and were practicing intensive home care for at least 18 months before the plaque was collected.

Human gingivitis thus appears to be initiated from teeth-adherent bacterial biofilms (plaques) by lysine decarboxylase, an enzyme endogenous in plants or bacteria but absent from vertebrates.

The present invention contemplates a vaccine for inducing antibodies that inhibit lysine decarboxylase therefore providing a simple, inexpensive therapy for periodontal disease in animals including humans and dogs, in which periodontal disease most resembles that of humans. Human and canine oral cavities possess two organisms that commonly make lysine decarboxylase, Eikenella corrodens and Staphylococcus epidermidis. In the present invention, the vaccine comprises an immunogenic composition that includes recombinant lysine decarboxylases derived from both of these bacteria (E. corrodens and S. epidermidis).

The immunogenic composition further includes a pharmaceutically acceptable carrier or excipient in which the proteins described herein above are disposed.

The present invention also includes methods of producing at least one of the immunogenic compositions described herein above. Such methods include providing a nucleotide sequence encoding at least one lysine decarboxylase, inserting such nucleotide sequence into a host cell, thereby creating a recombinant host cell encoding the lysine decarboxylase. The recombinant host cell is then grown under conditions that allow for expression of the lysine decarboxylase, and the lysine decarboxylase is then purified away from the recombinant host cell.

The method may further include the production of a second lysine decarboxylase in a manner similar to that described herein above for the first lysine decarboxylase. Nucleotide sequences encoding the first and second lysine decarboxylases may be inserted into a single recombinant host cell and expressed and purified together, or the first and second lysine decarboxylases may be inserted into separate host cells and expressed and purified separately.

The present invention further includes methods for inducing an immunogenic response in a subject. Such methods comprise administering at least one of the immunogenic compositions described above to the subject.

Methods of Vaccine Production

Recombinant lysine decarboxylase protein to be used as an immunogen in the vaccine can be produced, for example, using the pET plasmid system, developed at Brookhaven National Laboratory under contract with the U.S. Department of Energy. The system can express recombinant lysine decarboxylase in E. coli. This system is sold under the Novagen label, a subsidiary of EMD Biosciences Inc., San Diego, Calif. Other plasmid systems for producing recombinant lysine decarboxylase protein may also be used.

In a preferred embodiment, the coding sequence of the lysine decarboxylase gene from each organism (SEQ ID NO:1 for E. corrodens which encodes SEQ ID NO:2, and SEQ ID NO:3 for S. epidermidis which encodes SEQ ID NO:4) is cloned into plasmid pET11 and transformed into Novablue, an E. coli strain which cannot express the cloned gene because it does not possess T7 RNA polymerase. This procedure is necessary to eliminate potential host cell instability. Transient expression of lysine decarboxylase within the E. coli cytosol could remove lysine from the cytosol before it can be incorporated into proteins. Once established in the initial host, the recombinant pET11 is transferred to the expression host, E. coli strain BL21(DE3) containing the T7 RNA polymerase gene. Rapid expression can be induced by adding IPTG or lactose to the bacterial culture. Nucleotides numbered 1 and 2113 of the coding sequence for E. corrodens lysine decarboxylase (SEQ ID NO:1) correspond, respectively, to nucleotides 1217 and 3339 in GenBank nucleotide sequence U89166. Nucleotides numbered 1 and 1345 of the coding sequence for S. epidermidis lysine decarboxylase (SEQ ID NO:3) correspond, respectively, to the inverse complement of nucleotides 2376166 through 2374829 in GenBank nucleotide sequence AE015929.

Isolation of Genomic DNA and Cloning the Lysine Decarboxylase Genes

In one embodiment, genomic DNA can be purified from E. corrodens and S. epidermidis using a DNA purification mini-kit (e.g., QIAamp DNA Mini Kit; Qiagen, Valencia, Calif.), and the lysine decarboxylase gene fragment is copied using a high fidelity polymerase (e.g., KOD, HiFi, or Hot Start; Novagen, a subsidiary of EMD Biosciences, Inc., San Diego, Calif.) using primers (see Table I) terminating in NdeI and BamH1 sites. After digesting the product, the DNA fragment is separated by agarose gel electrophoresis and purified (Qiagen Gel Purification Kit). The vector plasmid DNA (pET11; Novagen) is purchased already digested. The sticky ends of the lysine decarboxylase gene are ligated into the plasmid's open NdeI/BamH1 site (Novagen DNA ligation kit No. 69838-3) and the recombined plasmid is used to transform E. coli strain Novablue. To avoid contamination, the DNA for each of these above steps is manipulated in a Biological Safety Cabinet.

TABLE I

| | | |
|---|---|---|
| Direction | Sequence | SEQ ID NO. |

| | | |
|---|---|---|
| Forward | GGGAATTCATATGAAGAACATC | 5 |
| Reverse | CAGTTGGATCCGCGTGGGTTAAGCTT | 6 |
| Forward | GGGAATTCATATGAAAAG | 7 |
| Reverse | CAGTTGGATCTTATTCATCCTT | 8 |

Primer Sequences for E. corrodens (SEQ ID NOs: 5 and 6) and S. epidermidis (SEQ ID NOs: 7 and 8)

Only transformed bacteria can grow on Luria Bertani (LB) agar containing 12.5 μg/ml tetracycline. To ensure that the bacterial cells have been transformed correctly, about 12 colonies are picked from the plate, grown in 5 ml LB medium, and the plasmid DNA isolated using a plasmid miniprep kit (Qiagen). The plasmid DNA is cut out using the NdeI and BamH1 sites, or PCR primers are made to copy the inserted region. The cut or copied DNA is sequenced to confirm the absence of mutations.

Expression and Detection of Recombinant Lysine Decarboxylase

A non-mutated insert of E. corrodens lysine decarboxylase gene can be transformed into E. coli expression strain BL21(DE3), for example. A transformed E. coli BL21(DE3) can be selected from an individual colony that grows on a Petri dish containing LB agar and 0.05 mg/ml ampicillin. A single colony is picked, inoculated into 100 ml of liquid LB containing 5 mg ampicillin and cultured at 30° C. for 18 hours with shaking at 220 rpm. An aliquot of approximately 15 ml of this culture is used to inoculate 1 liter of LB containing 50 mg of ampicillin. Cultures are grown at 37° C. with shaking at 180 rpm until an optical density of 0.8 at 600 nm is attained. At that time, expression is induced by addition of 120 mg of IPTG to each liter of culture.

The expressed, recombinant antigens either form insoluble inclusion bodies that comprise more than 50% of the total cell protein a few hours after induction, or large amounts of the antigen are secreted into the spent culture fluid. The spent culture fluid (500 ml) is centrifuged and the supernatant concentrated 20-fold using a Millipore membrane concentrator. The bacterial pellet is suspended in 50 mM Tris, 150 mM NaCl, pH 7.5 (TN buffer), and lysed by incubation with 6 mg lysozyme for 30 minutes, followed by freezing for 18 hours at −20° C. The concentrated culture fluid and lysate is then examined for a protein of the correct size by sodium dodecyl sulfate-polyacrylamide gel electrophoresis. The recombinant lysine decarboxylase from E. corrodens (SEQ ID NO:2) has a MW of about 80 kDa and that from S. epidermidis (SEQ ID NO:4) as a MW of about 52 kDa.

Solubilizing Inclusion Bodies

When cloned lysine decarboxylase protein is found in the lysate, MgCl₂ can be added to 1 mM concentration, and DNAse (1000 Kunitz units) added with stirring to remove DNA by incubating the mixture for 30 min. The volume can be expanded to 500 ml with TN buffer containing 0.1% Triton X-100 (TNT buffer), stirred for 30 minutes and the insoluble inclusion bodies pelleted by centrifugation. After washing by resuspension in TNT buffer with stirring for 1-2 hours, a sample of the pellet can be dissolved in sodium dodecyl sulfate and subjected to polyacrylamide gel electrophoresis to confirm that a protein of the correct size is a major component.

To solubilize the protein in the inclusion body pellet, three additional TNT washes are given and the pellet dissolved in 40 ml of 8 M urea, 1 mM EDTA, 1 mM glycine, 100 mM Tris base, 100 mM beta-mercaptoethanol (8 M urea buffer). Optical density at 280 nm is measured, and the volume expanded with 8 M urea buffer to achieve a final optical density of about 0.5. The pH of the solution can be adjusted to at least 10.0, and divided into four aliquots of 200 ml. Each 200 ml can be rapid-diluted into 4 liters of 20 mM Tris base, with rapid stirring. The pH can be adjusted immediately to 9.0, with 1.0 M HCl, and stored at 4° C. overnight. The following morning the diluted lysine decarboxylase solution is maintained at room temperature for 4-6 hours. The pH is adjusted to 8.5 and the flasks returned to 4° C. The same procedure is followed the next day with adjustment of pH to 8.0. The lysine decarboxylase solution in 4 4-liter flasks is then left at 4° C. for 2-3 weeks to solubilize. The approximately 16 liters total volume can be concentrated to 40 ml using ultra-filtration (Pellicon XL30 cartridge), and centrifuged at 140,000×g at 30 minutes in an ultracentrifuge pre-equilibrated to 4° C. The recovered supernatant is applied to a 2.5×100 cm column of Sephacryl S-300 equilibrated in 0.4 M urea, 20 mM Tris-HCl, pH 8.0. After elution at 30 ml/hour, the purified protein is salt exchanged into physiological saline.

Purifying the Recombinant Lysine Decarboxylase

Protein in the spent culture fluid can be concentrated 20-fold using a Pellicon XL30 cartridge with prefilter. The concentrate is passed over Sephacryl S-200 (Pharmacia) and purified from the cytoplasmic debris of E. coli BL21(DE3) by adherence at low ionic strength to an ion exchange column. DE Sephacel (Pharmacia Inc., NJ) is used to bind the acidic E. corrodens protein (isolectric pH 5.62) and carboxymethylcellulose (Pharmacia) is used to bind the basic S. epidermidis protein (isolectric pH 8.15). The respective proteins can be eluted by increasing the salt concentration. The major eluted protein peak is detected at 280 nm and examined after sodium dodecyl sulfate-polyacrylamide gel electrophoresis (SDS-PAGE). A sample of the concentrated culture fluid can be simultaneously examined as a control.

Purity is identified by Peptide Mass Fingerprinting from an in-gel digest using Matrix Assisted Laser Desorption/Ionization-Time Of Flight (MALDI-TOF) mass spectrometry at the EPSCoR Oklahoma Biotechnology Network Laser Mass Spectrometry Facility (section vii). The sequences of the resulting peptides are identified by searching a MALDI-TOF data base and the known encoded lysine decarboxylase sequences from GenBank (Accession numbers U89166 for E. corrodens and AE015929 for S. epidermidis).

Adjuvant

Although protein antigens usually induce a strong antibody response, immunity lasts much longer when given with an adjuvant, a viscous homogeneous material that provides a small region of highly concentrated antigen to stimulate the immune system. The adjuvant can be purchased as a sterile, pyrogen-free 3% aluminum hydroxide gel (Alhydrogel), for example, which is stable at room temperature, has a uniformly high adsorption capacity, especially in the absence of multivalent ions such as phosphate. Alhydrogel (Accurate Chemical & Scientific Corp., Westbury N.Y.) is stable for several years and adverse reactions to it have not been observed in dogs or humans.

Aliquots of each soluble recombinant or E. corrodens-derived or S. epidermidis-derived lysine decarboxylase protein (5 ml of 0.5 mg/ml) are transferred by pipette to a series of 12 test tubes, to which distilled water is added in amounts from 0 to 5.5 ml. Finally a 1 to 10 dilution of Alhydrogel in distilled water is added in amounts increasing from 0.5 to 6.0 ml. The total amount in each tube is then 11 ml. After cautious but rapid mixing and standing for some minutes, one of the tubes shows a marked flocculation. The floccules sediment and leave a clear supernatant. If the protein content of the solution is not ideal for adsorption under the conditions chosen, flocculation appears in the first or the last tube, and it is necessary to test with a more suitable dilution of protein or Alhydrogel. The amount of protein optimally adsorbed to Alhydrogel is estimated by measuring the protein concentration of the solution before and after adsorption using the Bradford protein reagent (Biorad Chemical Corp., Hercules, Calif.). The adsorbed protein suspension (vaccine) is stable for 3 to 4 years under refrigerated conditions or at room temperature, provided freezing is avoided.

Immunization—Example

Vaccine can be administered intramuscularly with 0.5 ml of Alhydrogel for example, containing 0.1 μg to 10 mg of total lysine decarboxylase antigen, and more preferably from 0.1 mg to 1 mg of antigen. Injections may be repeated again after three and six weeks for example, although other immunization schedules can be easily determined by persons of ordinary skill in the art.

Utility

The present invention in one embodiment is directed to a vaccine against lysine decarboxylases from E. corrodens and S. epidermidis, the vaccine thus comprising the entire lysine decarboxylase proteins thereof or immunogenic portions thereof.

The present invention also contemplates and describes herein novel primers (SEQ ID NOs:5-8) and their use in recognizing and amplifying all of or portions of the lysine decarboxylase genes described herein (SEQ ID NO:1, and SEQ ID NO:3, respectively).

More particularly, the present invention provides a vaccine composition which comprises an effective immunizing amount of an immunogenically active component selected from the group consisting of one or more whole, subunits or portions, of lysine decarboxylase from E. corrodens (SEQ ID NO:2) and one or more whole, subunits or portions, of lysine decarboxylase from S. epidermidis (SEQ ID NO:4) preferably, disposed in a pharmacologically acceptable carrier.

The present invention also provides a method for the prevention or amelioration of gingivitis and periodontitis in humans or animal subjects which comprises administering to said subject the vaccine composition as described herein disposed in a pharmacologically acceptable carrier to induce an immunogenic response effective against lysine decarboxylase in vivo.

One advantage of the vaccine described herein is to inhibit heart and other diseases. At present, oral bacterial products are absorbed into the blood stream and spread throughout the body, causing damage to the kidneys, heart, liver and brain. Giving the vaccine every 10 to 20 years to prevent periodontal disease in humans can prevent these periodontal disease-associated illnesses from also occurring.

As used herein, the term “immunogenic or immunogenically active” designates the ability to stimulate an immune response, i.e., to stimulate the production of antibodies, particularly humoral antibodies, or to stimulate a cell-mediated response. For example, the ability to stimulate the production of circulating or secretory antibodies or the production of a cell-mediated response in local mucosal regions, (e.g., intestinal mucosa), peripheral blood, cerebral spinal fluid or the like.

The effective immunizing amount of the immunogenic or immunogenically active component may vary and may be any amount sufficient to evoke an immune response and provide immunological protection against lysine decarboxylase.

At least one dosage unit per subject is contemplated herein as a vaccination regimen. In some embodiments, two or more dosage units may be especially useful. A dosage unit may typically be about 0.1 to 10 milliliters of vaccine composition, preferably about 0.5 to 5 milliliters, and even more preferably about 1 to 2 milliliters, with each dosage unit containing the previously described quantity of lysine decarboxylase protein or lysine decarboxylase component. The skilled artisan will quickly recognize that a particular quantity of vaccine composition per dosage unit, as well as the total number of dosage units per vaccination regimen, may be optimized, so long as an effective immunizing amount of the protein or a component thereof is ultimately delivered to the subject.

The lysine decarboxylase vaccine composition of the present invention may also contain one or more adjuvants or excipients. As used herein the term “adjuvant” refers to any component, which improves the body's response to a vaccine. The adjuvant will typically comprise about 0.1 to 50% vol/vol of the vaccine formulation of the invention, more preferably about 1 to 50% of the vaccine, and even more desirably about 1 to 20% thereof. Amounts of about 4 to 10% may be even more preferred. Adjuvants are well known in the art thus further description thereof herein is not deemed necessary.

In addition, the adjuvant may include one or more wetting or dispersing agents in amounts of about 0.1 to 25%, more preferably about 1 to 10%, and even more preferably about 1 to 3% by volume of the adjuvant. Particularly preferred as wetting or dispersing agents are non-ionic surfactants. Useful non-ionic surfactants include polyoxyethylene/polyoxypropylene block copolymers, especially those marketed under the trademark PLURONIC® and available from BASF Corporation (Mt. Olive, N.J.). Other useful nonionic surfactants include polyoxyethylene esters such as polyoxyethylene sorbitan monooleate, available under the trademark TWEEN 80®. It may be desirable to include more than one, e.g., at least two, wetting or dispersing agents in the adjuvant as part of the vaccine composition of the invention.

Other components of the adjuvant may include such preservative compounds as formalin and thimerosal in amounts of up to about 1% vol/vol of the adjuvant.

Pharmacologically acceptable carriers suitable for use in the vaccine composition of the invention may be any conventional liquid carrier suitable for pharmaceutical compositions, preferably a balanced salt solution, physiological saline, or other water-based solution suitable for use in tissue culture media. Other available carriers well known to those of ordinary skill in the art may also be utilized.

Additional excipients available and known to those of ordinary skill in the art may also be included in the vaccine composition according to the various embodiments heretofore described. For example, pH modifiers may be utilized.

The components of the vaccine composition of the invention as heretofore described, including the carrier, may be combined together using techniques known to those of ordinary skill in the art.

In one embodiment of the invention the immunogenically active component of the invention may be incorporated into liposomes using known technology such as that described in Nature, 1974, 252, 252-254 or the Journal of Immunology, 1978, 120, 1109-13. In another embodiment of the invention, the immunogenically active component of the invention may be conjugated to suitable biological compounds such as polysaccharides, peptides, proteins, polymers or the like, or a combination thereof.

In a preferred embodiment of the invention, the novel vaccine composition contemplated herein may be formulated in a dosage unit form as heretofore described to facilitate administration and ensure uniformity of dosage. Formulation may be effected using available techniques, such as those applicable to preparations of emulsions.

The novel vaccine composition contemplated herein may be administered, for example, parenterally, intramuscularly, subcutaneously, intraperitoneally, intradermally, orally, or intranasally. By way of non-limiting example, a typical treatment schedule or dosing regimen may include parenteral administration, preferably intramuscular injection of one dosage unit. At least two administrations may be preferred, for example a second dosage unit may be given about 3-5 weeks after the first innoculation. As heretofore set forth, a dosage unit will typically be within the range of about 0.1 to 10 milliliters of vaccine composition containing the previously described amounts of active and percentages of adjuvant and inactives set forth. A dosage unit within the range of about 0.5 to 5 milliliters is perhaps more preferred, with about 1 to 2 milliliter(s) being particularly preferred.

The subjects which may be treated with the lysine decarboxylase vaccine contemplated herein include, but are not limited to, mammals, including primates such as humans, chimpanzees, baboons, gorillas and orangutans, monkeys and lemurs; mustelids including minks; camelids, including camels, llamas, alpacas, and vicunas; felids including lions, tigers and domestic cats; canids including dogs; bovids including cattle; equids including horses; ovids including sheep and goats; suids including pigs; and cervids including deer, elk and moose.

Each of U.S. Pat. Nos. 6,103,220; 6,187,296; 6,576,435; and 6,974,700 is expressly incorporated herein by reference in its entirety.

### EXAMPLE

An Example is provided hereinbelow. However, the present invention is to be understood to not be limited in its application to the specific experimentation, results and laboratory procedures. Rather, the Examples are simply provided as one of various embodiments and are meant to be exemplary, not exhaustive.

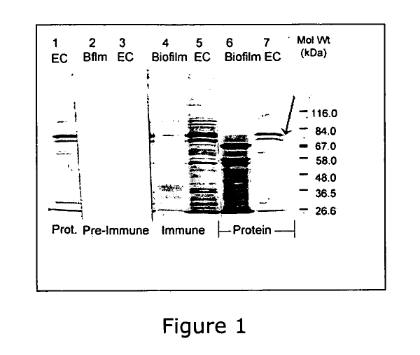

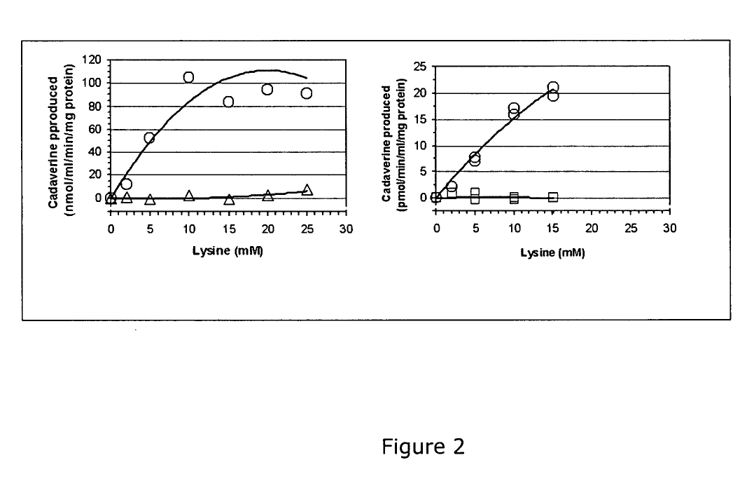

In one experiment, a cell-surface extract of E. corrodens containing about 25% lysine decarboxylase protein was used to immunize two goats (0.25 mg of protein from this E. corrodens extract after emulsification in 50% (v/v) Freunds complete adjuvant). The injections at 10-12 multiple subcutaneous sites on the back were repeated after 2 and 4 weeks using Freunds incomplete adjuvant. Goats were bled before immunization and two weeks after the last injection. The blood was clotted overnight at 0° C. to obtain pre- and post-immune serum. Enzyme activity was assayed in the presence or absence of 50% (v/v) serum, and E. corrodens extract was obtained as described elsewhere herein. Controls (no serum or pre-immune serum only) gave an asymptotically increasing amount of cadaverine as the lysine concentration increased from 0 to 25 mM. Maximal catalysis was 80-120 nmol cadaverine/min/mg protein/ml for E. corrodens extract and 0.03% of that for plaque pooled from the gingival region of teeth. Immune serum from both goats detected lysine decarboxylase on immunoblots (FIG. 1) and inhibited all activity in both E. corrodens and plaque extracts (FIG. 2), whereas pre-immune antibodies neither detected lysine decarboxylase nor inhibited its activity. The inhibiting power of goat antibodies was stable for at least 5 months.

Production of Antibody to S. epidermidis

Antibodies were made to an internal 118 amino acid portion of the S. epidermidis sequence, amino acids 102-219 believed to encode the pyridoxal phosphate cofactor binding site. A commercial vendor provided the antibodies, Strategic Diagnostics Inc. (SDI), a subdivision of Strategic Biosolutions, 111 Pencader Dr., Newark, Del. 19702. SDI analyzes this provided amino acid sequence using B-cell epitope identification algorithms to assess parameters such as hydrophilicity, charge, and surface probability. Another proprietary algorithm is used to identify regions of the protein with a high probability of generating antibodies using proprietary Genomic Antibody Technology™ (GAT) and also to identify sites of possible post-translational modification. The BLAST algorithm is used to assess potential cross-reactivity and immune tolerance. The protein sequence information is inserted into SDI's proprietary plasmid vector which is then introduced into rabbits. Cells of the host animal take up the plasmid, synthesize the polypeptide and secrete it, causing it to be recognized by the immune system. Strategic Biosolutions provided 80 ml of rabbit antiserum that reacts strongly to this polypeptide sequence for use in detecting the S. epidermidis antigen.

### Preparation of Genomic DNA Encoding Lysine Decarboxylase

Recombinant lysine decarboxylase protein to be used as an immunogen in the vaccine can be produced, for example, using the pET plasmid system, developed at Brookhaven National Laboratory under contract with the U.S. Department of Energy. The system can express recombinant lysine decarboxylase in E. coli if induced by adding IPTG or lactose to the bacterial culture. This system is sold under the Novagen label, a subsidiary of EMD Biosciences Inc., San Diego, Calif. Other plasmid systems for producing recombinant lysine decarboxylase protein may also be used.

The E. corrodens lysine decarboxylase gene (SEQ ID NO:1, nucleotides numbered 1 and 2113 of the coding sequence) corresponds, respectively, to nucleotides 1217 and 3339 in GenBank nucleotide sequence U89166 that encodes polypeptide SEQ ID NO:2. The S. epidermidis lysine decarboxylase gene (SEQ ID NO:3, nucleotides numbered 1 and 1345 of the coding sequence) corresponds, respectively, to the inverse complement of nucleotides 2376166 through 2374829 in GenBank nucleotide sequence AE015929 that encodes polypeptide SEQ ID NO:4.

In a preferred embodiment, the respective coding sequences of the lysine decarboxylase gene from each organism (SEQ ID NO:1 for E. corrodens and SEQ ID NO:3 for S. epidermidis) are modified to change the codons for each amino acid to preferred E. coli codons. This revised nucleotide sequence of each gene (SEQ ID NO:5 and SEQ ID NO:6) is artificially synthesized with added NdeI and BamH1 restriction site extensions at each end for ligation into pET11a. The recombinant pET11a is transformed into E. coli strain ER2566 to ensure that there is no host cell instability and then transferred to the expression host, E. coli strain BL21(DE3) containing the T7 RNA polymerase gene. These procedures are commercially available from Genscript Corp., 120 Centennial Ave. Piscataway, N.J.

Expression and Detection of Recombinant Lysine Decarboxylase

A pET11a transformed E. coli BL21(DE3) can be selected from an individual colony that grows on a Petri dish containing LB agar and 0.05 mg/ml ampicillin. A single colony is picked, inoculated into 3 ml of liquid LB containing 0.3 mg ampicillin (0.1 mg/ml) and cultured at 37° C. for 16 hours with shaking at 200 rpm. This culture added to inoculate 25 ml of fresh LB containing 2.5 mg of ampicillin. Alternatively the 3 ml culture can be inoculated into a liter or more of LB/ampicillin if commercial amounts of recombinant antigen are required. Growth at 37° C. with aeration, for example shaking a 25 ml flask at 200 rpm, is permitted until an optical density of 0.7±0.1 at 600 nm is attained (about 3 h). Recombinant antigen expression is then induced by adding 12.5 μmol of IPTG for 3 h. Un-induced cultures have no added IPTG.

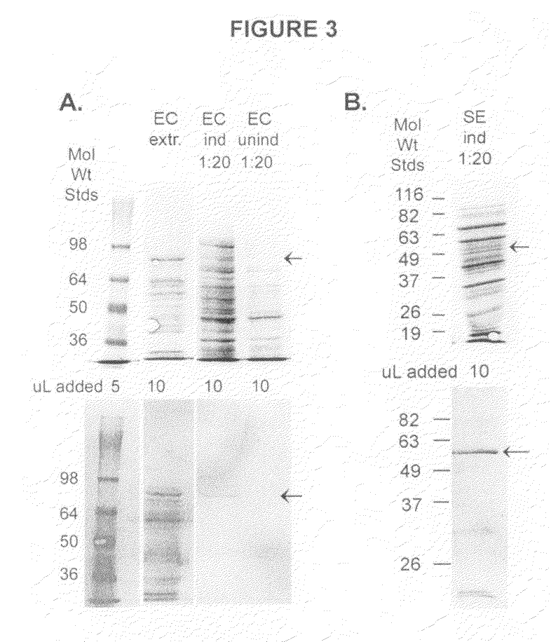

After induction, 1 ml aliquots are removed from the large culture into microfuge tubes and centrifuged at 12000 rpm for 2 min at 4°. The liquid supernatant is removed and 50 μL of cold (4°) 25 mM Tris-Cl, pH 8.0 added to resuspend each pellet. After centrifugation and supernatant removal as before, the pellets are frozen for at least 30 min at −80°. The presence of expressed lysine decarboxylase in the pellet is detected by sodium dodecyl polyacrylamide gel electrophoresis (SDS-PAGE). The induced E. coli pellet is removed from the freezer, thawed (1 or 2 minutes) and resuspended in 40 μL lysis buffer (0.05 M Tris, pH 8.0, 5 mM MgCl₂, 50 μg/mL RNase A, 10 μg/mL DNase I). Lysozyme (1 μL of 50 mg/mL) is added to the suspension, which dissolves when incubated at room temperature for 15 minutes. An equal volume of sample buffer (1.0 mL 0.5M Tris-Cl, pH 6.8 containing 0.80 mL glycerol, 1.6 mL 10% (w/v) SDS, 0.4 mL β-mercaptoethanol, 0.2 mL 0.05% Bromophenol blue) is then added and diluted further if necessary. FIG. 3A shows typical expression of E. corrodens lysine decarboxylase in the E. coli pellets from IPTG induced, but not un-induced cultures. FIG. 3B shows a similar result for the lysine decarboxylase from S. epidermidis. As expected, the recombinant lysine decarboxylase from E. corrodens (SEQ ID NO:2) has a MW of about 80 kDa and that from S. epidermidis (SEQ ID NO:4) as a MW of about 52 kDa.

Purification of Recombinant Lysine Decarboxylase

The expressed, recombinant antigens either form insoluble inclusion bodies that comprise more than 50% of the total cell protein a few hours after induction, or large amounts of the antigen are secreted into the spent culture fluid. The spent culture fluid (500 ml) is centrifuged and the supernatant concentrated 20-fold using a Millipore membrane concentrator. The bacterial pellet is suspended in 50 mM Tris, 150 mM NaCl, pH 7.5 (TN buffer), and lysed by incubation with 6 mg lysozyme for 30 minutes, followed by freezing for 18 hours at −20° C. The concentrated culture fluid and lysate is then examined for a protein of the correct size by sodium dodecyl sulfate-polyacrylamide gel electrophoresis (FIGS. 3A and 3B).

Immunization—Example

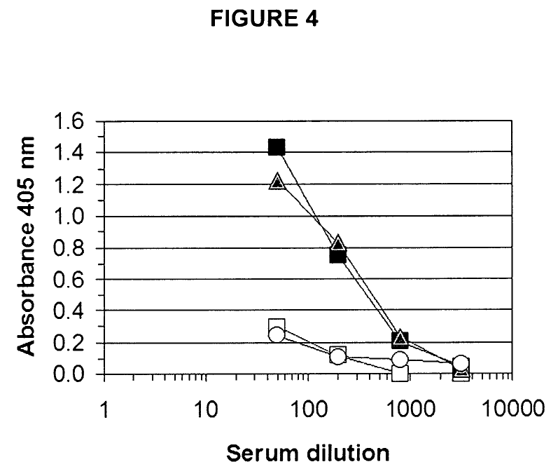

Vaccine can be administered intramuscularly with 1.0 ml of Rehydrogel for example, containing 0.1 μg to 10 mg of lysine decarboxylase antigen, and more preferably from 0.1 mg to 1 mg of antigen. Injections may be repeated again after three and six weeks for example, although other immunization schedules can be easily determined by persons of ordinary skill in the art. Dogs were injected with 1 ml of 15% Rehydragel slurry (recommended by the manufacturer) with or without 0.2 mg protein from E. corrodens extract. FIG. 4 shows that two dogs injected with the E. corrodens extract protein produced antibodies in serum diluted 200 fold whereas two control dogs produced very little antibodies.

The present invention is not to be limited in scope by the specific embodiments described herein, since such embodiments are intended as but single illustrations of one aspect of the invention and any functionally equivalent embodiments are within the scope of this invention. Indeed, various modifications of the methods and compositions of the invention in addition to those shown and described herein will become apparent to those skilled in the art from the foregoing description.

### REFERENCES

The following references, to the extent that they provide exemplary procedural or other details supplementary to those set forth herein, are specifically incorporated herein by reference.
Hardham J, Dreier K, Wong J, Sfintescu C, Evans R T (2005a). Pigmented-anaerobic bacteria associated with canine periodontitis. Vet Microbiol 106(1-2): 119-128.
Hardham J, Reed M, Wong J, King K, Laurinat B, Sfintescu C et al. (2005b). Evaluation of a monovalent companion animal periodontal disease vaccine in an experimental mouse periodontitis model. Vaccine 23(24):3148-3156.
Levine M, Progulske-Fox A, Denslow N D, Farmerie W G, Smith D M, Swearingen W T et al. (2001). Identification of lysine decarboxylase as a mammalian cell growth inhibitor in Eikenella corrodens: possible role in periodontal disease. Microb Pathog 30(4):179-192.
Li J, Helmerhorst E J, Leone C W, Troxler R F, Yaskell T, Haffajee A D et al. (2004). Identification of early microbial colonizers in human dental biofilm. J Appl Microbiol 97(6): 1311-1318.
Lindhe J, Hamp S E, Loe H (1973). Experimental periodontitis in the beagle dog. Int Dent J 23(3):432-437.
Lindhe J, Hamp S E, Loe H (1975). Plaque induced periodontal disease in beagle dogs. A 4-year clinical, roentgenographical and histometrical study. J Periodontal Res 10(5):243-255.
Listgarten M A (1986). Pathogenesis of periodontitis. J Clin Periodontol 13(5):418-430.
Loe H, Holm-Pedersen P (1965). Absence and presence of fluid from normal and inflamed gingivae. Periodontics 3:171-177.
Sandmeier E, Hale T I, Christen P (1994). Multiple evolutionary origin of pyridoxal-5′-phosphate-dependent amino acid decarboxylases. Eur J Biochem 221(3):997-1002.
Socransky S S, Haffajee A D (2005). Periodontal microbial ecology. Periodontol 2000 38:135-187.
Socransky S S, Haffajee A D, Cugini M A, Smith C, Kent R L, Jr. (1998). Microbial complexes in subgingival plaque. J Clin Periodontol 25(2):134-144.

What is claimed is:

1. An immunogenic composition for inducing an immune response against lysine decarboxylase for treating or preventing periodontitis in a subject, comprising:
Eikenella corrodens lysine decarboxylase or an immunogenic portion thereof;
Staphylococcus epidermidis lysine decarboxylase or an immunogenic portion thereof; and
a pharmaceutically acceptable carrier or excipient.

2. The immunogenic composition of claim 1, wherein the E. corrodens lysine decarboxylase has the sequence SEQ ID NO:2.

3. The immunogenic composition of claim 1, wherein the S. epidermidis lysine decarboxylase has the sequence of SEQ ID NO:4.

4. The immunogenic composition of claim 1, further comprising an adjuvant.

5. A method for inducing an immunogenic response in a subject, comprising:
administering the immunogenic composition of claim 1 to the subject.

6. The method of claim 5, wherein the subject is selected from the group consisting of mammals, primates, humans, chimpanzees, baboons, gorillas, orangutans, monkeys, lemurs, mustelids, minks, camelids, camels, llamas, alpacas, vicunas, felids, lions, tigers, domestic cats, canids, dogs, bovids, cattle, equids, horses, ovids, sheep, goats, suids, pigs, cervids, deer, elk and moose.

7. The method of claim 5, wherein the immunogenic composition is administered by at least one of parenterally, intramuscularly, subcutaneously, intraperitoneally, intradermally, orally, arterially, rectally, vaginally, intralymphnodally, and intranasally.
